(19) United States
(12) Patent Application Publication
(10) Pub. No.: US 2003/0220267 A1
(43) Pub. Date: Nov. 27, 2003

(54) Borinic acid protease inhibitors

(76) Inventors: Donald S. Matteson, Moscow, ID (US); Levente Fabry-Asztalos, Madison, WI (US); William H. Prusoff, Branford, CT (US); Arman Pivazyan, Woodbridge, CT (US)

Correspondence Address: SCHWEGMAN, LUNDBERG, WOESSNER & KLUTH, P.A., P.O. BOX 2938, MINNEAPOLIS, MN, 55402, US

(21) Appl. No.: 10/364,930

(22) Filed: Feb. 11, 2003

Related U.S. Application Data
(63) Continuation of application No. PCT/US02/26300, filed on Aug. 16, 2002.
(60) Provisional application No. 60/312,725, filed on Aug. 16, 2001.
(60) Provisional application No. 60/315,298, filed on Aug. 27, 2001.
(60) Provisional application No. 60/322,321, filed on Sep. 12, 2001.

(51) Int. Cl. A61K 38/06; C07K 7/06

(52) U.S. Cl. 514/18; 530/330

(57) ABSTRACT

The invention provides compounds of formula I:

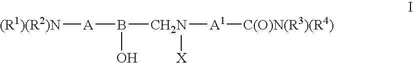

wherein R¹-R⁴, A, A¹, and X have any the values described in the specification, as well as pharmaceutical compositions comprising such compounds, and methods of inhibiting proteases with such compounds. The invention also provides synthetic intermediates and processes useful for preparing compounds of formula (I).

### PRIORITY OF INVENTION

This application is a continuation under 35 U.S.C. 111(a) of PCT/US02/26300, filed 16 Aug. 2002, which claims priority from U.S. Provisional Application No. 60/312,725, filed 16 Aug. 2001; and from U.S. Provisional Application No. 60/315,298, filed 27 Aug. 2001; and from U.S. Provisional Application No. 60/322,321, filed 12 Sep. 2001; all of which are incorporated by reference herein.

### BACKGROUND

Human immunodeficiency virus 1 (HIV-1) protease is a member of the aspartyl-protease family of enzymes. It is necessary for the posttranslational processing of the HIV-1 polyprotein gene products of Pr55^{gag} and Pr160^{gag-pol}. The scission of these fusion polyproteins by the HIV-1 encoded protease leads to the release of mature viral structural proteins (p6, p7, p17, and p24) and replication enzymes (reverse transcriptase, protease itself, and integrase). Inhibition of the HIV-1 protease results in the production of noninfectious viral particles. See Wlodawer, A. et al., Annu. Rev. Biophys. Biomol. Struct., 27, 249-284 (1998); Wlodawer, A., Annu. Rev. Bichem., 62, 543-585 (1993); De Clerq, E., J. Med. Chem., 38, 2491-2517 (1995); and Kohl, N. E. et al., Proc. Natl. Acad. Sci. USA, 85 4686-4690 (1988).

HIV-1 protease is a C2 symmetric homodimeric protein with a molecular weight of 22 kD. Teach monomer is formed by 99 residues organized in β strands, β sheets, β chains, and broad loops. The homodimeric protein contains only one active site through which the enzyme catalyzes the hydrolysis of specific peptide bonds. The active site triad (Asp 25, Thr 26, Gly 27) is located in the loop region of the enzyme. See Wlodawer, A., Annu. Rev. Bichem., 62, 543-585 (1993). The first crystal structure of the enzyme was solved in 1989.

HIV inhibition from the point of view of drug design has been approached by targeting HIV protease, reverse transcriptase, integrase and CD 4 and CCR 5 cellular receptor proteins. See Wlodawer, A. et al., Annu. Rev. Biophys. Biomol. Struct., 27, 249-284 (1998); Litterst, C., Antiviral Chemotherapy, 397-404 (1996); and Ashom, B. et al., Proc. Natl. Acad. Sci. USA, 87, 8889-8893 (1990). Currently, six HIV-1 protease competitive inhibitors, Ritonavir (Norvir), Nelfinavir (Viracept), Saquinavir (Invirase and Fortovase), Indinavir (Crixivan), Amprenavir (Agenerase), and Lopinavir (Kaletra) are in clinical use. There are also many peptide and non-peptide based inhibitors under advanced clinical trials (ex. U-140690, VX-478, KNI-272, DMP-450). See Wlodawer, A. et al., Annu. Rev. Biophys. Biomol. Struct., 27, 249-284 (1998). Furthermore, there are many, both symmetric and non-symmetric structures under intense study and development. Although, non-peptide based inhibitors have inhibition constants in the range of 10⁻⁹ to 10⁻¹⁰ M, these compounds are not ideal because they usually have poor aqueous solubility, poor oral bioavailability, brief duration of action, and rapid elimination.

Currently, the clinical approach for the therapy of AIDS utilizes the co-administration of two reverse transcriptase inhibitors with one protease inhibitor (usually referred to as cocktail or combination therapy). Cocktail therapy extensively reduces viremia to very low levels. However, in 30-50% of patients antiviral therapy is ineffective due to resistance development and/or patient non-adherence. Furthermore, in many patients, side effects associated with these drugs pose serious problems. Some of the side effects are diabetes, high blood pressure, and heart disease. Because of the above mentioned resistance development and toxicity there is a urgent need for the development of more efficient drugs with different resistance profiles, with decreased toxicity and with more than one type of inhibitory action.

Most of the current drug discovery approaches for HIV-1 protease inhibition are based on the synthesis of peptide analogs in which the scissile amide bond of the viral polyproteins (most frequently Phe-Pro) has been replaced by a nonhydrolyzable isostere with tetrahedral geometry, like hydroxyethylene, phosphinate, hydroxyethylamine, and dihydroxyethylene. See Wlodawer, A., Pharmacotherapy, 14, 104-205 (1994). The optimum competitive inhibitors are peptidomimetics that comprise the two residues proceeding and the two residues following the scissile bond, with large hydrophobic side chains (ex. Phe, Leu, Ile) in the central position. Moreover, more rigid inhibitors that are more tightly constrained (packed) are preferred for entropic reasons. Typically competitive inhibitors are bound in the HIV-1 protease active site in an extended conformation and all the contacts between the main chain of the inhibitor and the protease are very similar. See Wlodawer, A., Pharmacotherapy, 14, 104-205 (1994).

The HIV-1 protease can also be made inactive by associative inhibitors, which prevent dimerization. Since it has been shown that the N and C terminal amino acids of the HIV-1 protease have an important role in the formation of an active dimeric enzyme, the corresponding peptidomimetics of those residues can interfere witn the dimerization process.

In many previous studies boronic a cid derivatives were proven to be good inhibitors of enzymes, especially serine proteases with Kᵢ values ranging from 10⁻¹⁰ to 10⁻¹² M. See Amiri, P. et al., Archives of Biochemistry and Biophysics, 234, 531-536 (1984); Bachovchin, W. W. et al., The Journal of Biological Chemistry, 265 3738-3743 (1990); Bone, R. et al., Biochemistry, 28, 7600-7609 (1989); Duncan, K. et al., Biochemistry, 28, 3541-3549 (1989); Kettner, C. et al., The Journal of Biological Chemistry, 265, 18289-18297 (1990); Kettner, C. A. et al., The Journal of Biological Chemistry, 259, 15106-15114 (1984); Koehler, K. A. et al., Biochemistry, 10, 2477-2483 (1971); Koehler, K. A. et al., Biochemistry, 13, 5345-5350 (1974); Lindquist, R. N. et al., J. Am. Chem. Soc., 99, 6435-6436 (1977); Matteson, D. S. et al., J. Am. Chem. Soc., 103, 5241-5242 (1981); Shenvi, A. B., Biochemistry, 25, 1286-1291 (1986); and Wityak, J. et al., J. Org. Chem., 60, 3717-3722 (1995). Peptide modification by incorporating a boronic acid increases the affinity of peptide inhibitors toward proteases and other enzymes. As result, stronger complexes with the active site of the enzymes are formed. Peptide boronic acids, peptide analogues in which the C-terminus carboxy moiety is replaced by dihydroxyboron, are very good inhibitors of different proteases by mimicking the formation of high-energy tetrahedral intermediates on the reaction pathway for peptide-bond hydrolysis.

N-Acetyl-L-phenylalamine's α-amido boronic acid analogue was first synthesized in 1981. It proved to be a good inhibitor of chymotrypsin with Kᵢ of 2.1 μM at pH 7.5. See Matteson, D. S. et al., J. Am. Chem. Soc., 103, 5241-5242 (1981). Subsequently, other groups, both in academia and industry, have synthesized a variety of boronic acid derivatives, many of which proved to be good inhibitors of enzymes. A few examples of enzymes inhibited by boronic acids are: chymotrypsin, esterase, thrombin, IgA1 proteinase, elastase, α-lytic protease, acetylcholinesterase, bacillus stearosthermophilus alanine racemase, and aminopeptidases. See Amiri, P. et al., Archives of Biochemistry and Biophysics, 234, 531-536 (1984); Bachovchin, W. W. et al., The Journal of Biological Chemistry, 265 3738-3743 (1990); Bone, R. et al., Biochemistry, 28, 7600-7609 (1989); Duncan, K. et al., Biochemistry, 28, 3541-3549 (1989); Kettner, C. et al., The Journal of Biological Chemistry, 265, 18289-18297 (1990); Kettner, C. A. et al., The Journal of Biological Chemistry, 259, 15106-15114 (1984); Koehler, K. A. et al., Biochemistry, 10, 2477-2483 (1971); Koehler, K. A. et al., Biochemistry, 13, 5345-5350 (1974); Matteson, D. S. et al., J. Am. Chem. Soc., 103, 5241-5242 (1981); Matteson, D. S. et al., Organometallics, 3, 1284-1288 (1984); Shenvi, A. B., Biochemistry, 25, 1286-1291 (1986); and Wityak, J. et al., J. Org. Chem., 60, 3717-3722 (1995).

Additionally, many kinetic (Kettner, C. A. et al., Biochemistry, 27, 7682-7688 (1988); and Gutheil, W. G. et al., Biochemistry, 32, 8723-8731 (1993)), X-ray crystallography (Bone, R. et al., Biochemistry, 26, 7609-7614 (1987); and Bone, R. et al., Biochemistry, 28, 7600-7609 (1989)), and NMR (Sundmeier, J. L. et al., Biochemistry, 33, 12427-12438 (1994); and Tsilikomas, E. et al., Biochemistry, 32, 12651-12655 (1993)) studies have been carried out with boronic acid inhibitors.

### SUMMARY OF THE INVENTION

Applicant has discovered a series of borinic acid compounds that are inhibitors of mammalian proteases, in particular, HIV-1 protease. Accordingly, the invention provides a compound of formula (I):

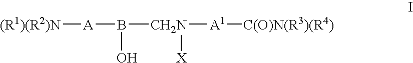

wherein A is the residue of an amino acid, A¹ is the residue of an amino acid, R¹, X and R³ are individually H, or a C₁-C₁₂ organic substituent, R² and R⁴ are individually R¹ or are (R¹)(R³)N[—A¹—C(O)]ₙ, wherein A¹ is preferably A, and n is 1-25 or N together with R¹ and R² or R³ and R⁴ are a 5-7 membered heterocyclic ring, containing 1-3 N(R⁵), S or nonperoxide O, wherein R⁵ is absent or is R¹, or a pharmaceutically acceptable salt thereof.

The invention also provides a compound of formula II:

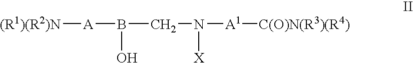

wherein A is the residue of a naturally occurring or synthetic alpha-amino acid, A¹ is the residue of a naturally occurring or synthetic amino acid of N—A or N(X)A¹ independently represent a heterocyclic ring; R¹, X and R³ are individually H. (C₁-C₆)alkyl, (C₆-C₁₀)aryl, (C₅-C₁₀)heteroaryl, (C₈-C₁₂)aralkyl, (C₈-C₁₂)heteroaralkyl, (C₅-C₁₀)cycloalkyl, (C₇-C₁₂)cycloalkylalkyl, (C₂-C₇)acyl or (C₁-C₄)alkyl)₃Si, R² and R⁴ are individually R¹ or (R¹)(R³)N[—A—C(O)]ₙ— wherein n is 1-25, preferably 2-15, most preferably 3-10 or, together with N, R¹ and R² or R³ and R⁴ are a 5-7 membered heterocyclic ring containing 1-3 N(R⁵), S or nonperoxide O, wherein R⁵ is absent, or is R¹ or a pharmaceutically acceptable salt thereof.

The invention also provides a pharmaceutical composition comprising a compound of formula I or II, or a pharmaceutically acceptable salt thereof, in combination with a pharmaceutically acceptable diluent or carrier.

Additionally, the invention provides a therapeutic method for preventing or treating a pathological condition or symptom in a mammal, such as a human, wherein the activity of a mammalian protease is implicated and antagonism of its action is desired comprising administering to a mammal in need of such therapy, an effective amount of a compound of formula I or II, or a pharmaceutically acceptable salt thereof.

The invention provides a compound of formula I or II for use in medical therapy (e.g. for use in treating viral infections or abnormal cellular proliferation), as well as the use of a compound of formula I or II for the manufacture of a medicament useful for the treatment of viral infections or abnormal cellular proliferation, such as cancer, in a mammal, such as a human.

A compound of the invention can be used as a tool to identify potential therapeutic agents for the treatment of diseases or conditions associated with unwanted enzyme activity, by contacting said agents with said borinic acid-enzyme complexes, and measuring the extent of displacement of the borinic acid and/or binding of the agent.

The invention also provides a compound prepared according to a synthetic method as described herein (e.g. a compound prepared as described in Example 1, 2, or 3 hereinbelow).

The invention also provides processes and intermediates disclosed herein that are useful for preparing compounds of formula I or II or salts thereof.

### BRIEF DESCRIPTION OF THE FIGURES

FIGS. 1-9 illustrate the synthesis of compounds of the invention.

FIG. 10 illustrates cyclic borinic acid structures

FIG. 11 illustrates the synthesis of compounds of the invention.

### DETAILED DESCRIPTION

As used herein, the term “organic substituents” refers to a wide range of chemical moieties comprising at least one carbon atom, such as those substituents described for compounds of formula II. Such substituents include alkyl (including alkenyl and alkynyl), aryl, aralkyl, heteroaryl, heteroaralkyl, heterocyclyl, cycloalkyl, cycloalkyl(alkyl), aryl and tris(alkyl)silyl. In turn, these organic substituents may include 1-3 substituents including OH, CN, NO₂, N(R¹)(R²), S(R¹), OR¹, —C(═O)OR¹, CO₂N(R¹)(R²), halo, R¹C(═O)O— and the, like, wherein substituents other than alkyl can be substituted by alkyl.

The following definitions are used, unless otherwise described: halo is fluoro, chloro, bromo, or iodo. Alkyl, alkoxy, alkenyl, alkynyl, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

It will be appreciated by those skilled in the art that compounds of tile invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine protease inhibitory activity using the standard tests described herein, or using other similar tests which are well known in the art.

Specific and preferred values listed below for radicals, substituents, and ranges, are for illustration only; they do not exclude other defined values or other values within defined ranges for the radicals and substituents

Specifically, A is the residue of an alpha-amino acid and A¹ is the residue of an alpha-amino acid.

Specifically, A is the residue of an alpha-amino acid or A¹ is the residue of an alpha-amino acid.

Specifically, the C₁-C₁₂ organic substituent is (C₁-C₆)alkyl, (C₆-C₁₀)aryl, (C₅-C₁₀)heteroaryl, (C₈-C₁₂)heteroaralkyl, (C₅-C₁₀)cycloalkyl, (C₂-C₄)acyl or ((C₂-C₄)alkyl)₃Si.

Specifically, A is the residue of a naturally occurring alpha-amino acid.

Specifically, A¹ is the residue of a naturally occurring alpha-amino acid.

Specifically, R¹, X, R³ and R⁴ are H.

Specifically, R¹, X, R³ or R⁴ is H.

Specifically, X is (C₁-C₆)alkyl.

Specifically, X is methyl.

Specifically, n is 2-15

Specifically, n is 3-10.

Specifically, each of the organic substituents is independently selected from (C₁-C₆)alkyl, (C₂-C₆)alkenyl, (C₂-C₆)alkynyl, (C₆-C₁₀)aryl, (C₁-C₁₀)heteroaryl, (C₈-C₁₂)aralkyl, (C₂-C₁₂)heteroaralkyl, (C₅-C₁₀)cycloalkyl, (C₇-C₁₂)cycloalkyl(alkyl), (C₂-C₇)acyl and (C₁-C₄)alkyl)₃Si; wherein each organic substituent is optionally substituted with one or more (e.g. 1, 2, 3, or 4) substituents independently selected from OH, CN, NO₂, N(R^{a})(R^{b}), S(R^{a}), OR^{a}, —C(═O)OR^{a}, CO₂N(R^{a})(R^{b}), halo, and R^{a}C(═O)O—; and wherein each R^{a} and R^{b} is independently hydrogen or (C₁-C₆)alkyl.

Specifically, A¹ is the residue of an amino acid.

Specifically, R¹ is H and R² is (C₂-C₇)acyl.

Specifically, R¹ is H and R² is acetyl.

Specifically, N(X)A¹ is pyrrolidin-2-yl or 4-hydroxyl-2-pyrrolidinyl.

Specifically, A is CH(phenyl).

Specifically, (R¹)(R³)N[—A—C(O)]ₙ is CH₃C(O)-Ser-Leu-Asn- or Ac-Thr-Leu-Asn and R¹ is H.

Specifically, R³ and R⁴ are H.

Specifically, N(X)A¹—C(O)N(R³)R⁴) is

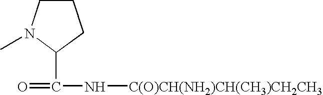

A specific compound of the invention is CH₃C(O)-Thr-Leu-Asn-Phe-B(OH)CH₂Pro-Ile; or a pharmaceutically acceptable salt thereof.

Another specific compound of the invention is CH₃C(O)-Leu-Asn-Phe-B(OH)CH₂-Pro-Ile; or a pharmaceutically acceptable salt thereof.

Another specific compound of the invention is CH₃C(O)-Asn-Phe-B(OH)CH₂-Pro-Ile; or a pharmaceutically acceptable salt thereof.

Another spcific compound of the invention is CH₃C(O)-ser-Leu-Asn-Phe B(OH)CH₂ProNH₂; or a pharmaceutically acceptable salt thereof.

Another specific compound of the invention is CH₃C(O)-Thr-Leu-Asn-Phe B(OH)CH₂ProNH₂; or a pharmaceutically acceptable salt thereof.

Another specific compound of the invention is CH₃C(O)-Phe B(OH)CH₂ProNH₂; or a pharmaceutically acceptable salt thereof.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1- hexynyl, 2-hexynyl, 3-hexynyl, 4-hexynyl, or 5-hexynyl; (C₂-C₇)acyl can be acetyl, propanoyl or butanoyl; halo(C₁-C₆)alkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; hydroxy(C₁-C₆)alkyl can be hydroxymethyl, 1-hydroxyethyl, 2-hydroxyethyl, 1-hydroxypropyl, 2-hydroxypropyl, 3-hydroxypropyl, 1-hydroxybutyl, 4-hydroxybutyl, 1-hydroxypentyl, 5-hydroxypentyl, 1-hydroxyhexyl, or 6-hydroxyhexyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)alkylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyi, isoquinolyl (or its N-oxide) or quinolyl (or its N-oxide).

A or A¹ can comprise the residues, e.g., the portion of the molecule other than the carboxylic acid and the amino group, preferably the alpha-amino group, of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, omithine, citruline, α-methyl-alanine, para-benzoylphenylalanine, phenylglycine, propargylglycine, sarcosine, and tert-butylglycine). A or A¹ also comprise residues of natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein).

Most preferably, each of A and A¹ is individually the residue of an α-amino acid, most preferably, a residue of a naturally occurring L-amino acid, such as the alkylidenyl or substituted alkylidenyl residues derived from glycine (Gly) (—CH₂—), alanine (Ala) (CH₃CH—), serine (Ser) (CH₂(OH)CH—), threonine (Thr) (CH₃CHOHCH—), valine (Val) (CH₃CH(CH₃)CH—), leucine (Leu) (—CH₃CH(CH₃)CH₂CH—), isoleucine (Ile) (CH₃CH₂CH(CH₃)CH—), cysteine (CySH) (CH₂(SH)CH—), cystine (CyS-SCy) [—SCH₂CH—]₂, phenylalanine (Phe) (PhCH₂CH—), tyrosine (Tyr) (4-HOPhCH₂CH—), proline (Pro) (pyrrolidin-2-yl), hydroxyproline (4-hydroxy-2-pyrrolidinyl), tryptophan (Trp) ((indol-3-yl)CH₂CH—), aspartic acid (Asp) (HOOCCH₂CH—), glutamic acid (HOOCCH₂CH₂CH—); histidine (His) ((imidazol-3-yl)CH₂CH—), lysine (Lys) (H₂N—(CH₂)₄CH—), or arginine (Arg) (H₂NC(═NH)(CH₂)₃CH—). Free CO₂H, NH₂, OH, or SH groups on A or A¹ groups can optionally be protecteed with suitable protecting groups (Z) such as tBoc, Cbz, acyl, benzyl, silyl, hemiacetals, or alkyl.

(R¹)(R³)N[—A—C(O)]ₙ— describes a sequence of 2 to 25 amino acids (e.g. as defined hereinabove) or peptidyl residues. The sequence may be linear or cyclic. For example, a cyclic peptide can be prepared or may result from the formation of disulfide bridges between two cysteine residues in a sequence. A peptide can be linked to the remainder of a compound of formula I or II through the carboxy terminus as shown, the amino terminus, or through any other convenient point of attachment, such as, for example, through the sulfur of a cysteine. Preferably a peptide comprises 3 to 20, or 3 to 10 amino acids. Peptide derivatives can be prepared as disclosed in U.S. Pat. Nos. 4,612,302; 4,853,371; and 4,684,620, or as described in the Examples hereinbelow. Peptide sequences specifically recited herein are written with the amino terminus on the left and the carboxy terminus on the right.

Processes for preparing compounds of formula I are provided as further embodiments of the invention and are illustrated by the following procedures.

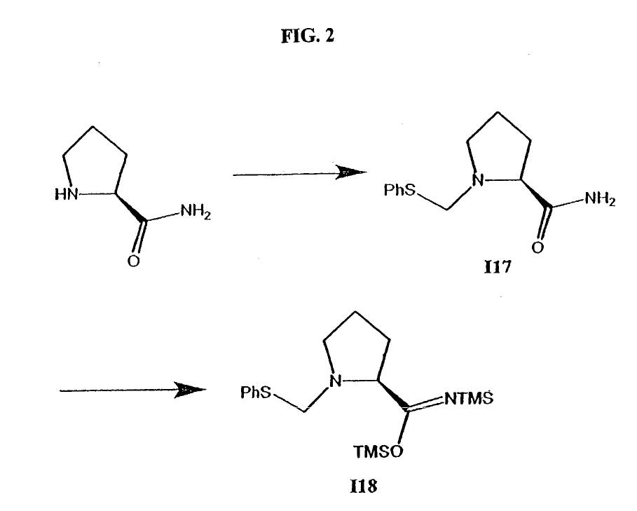

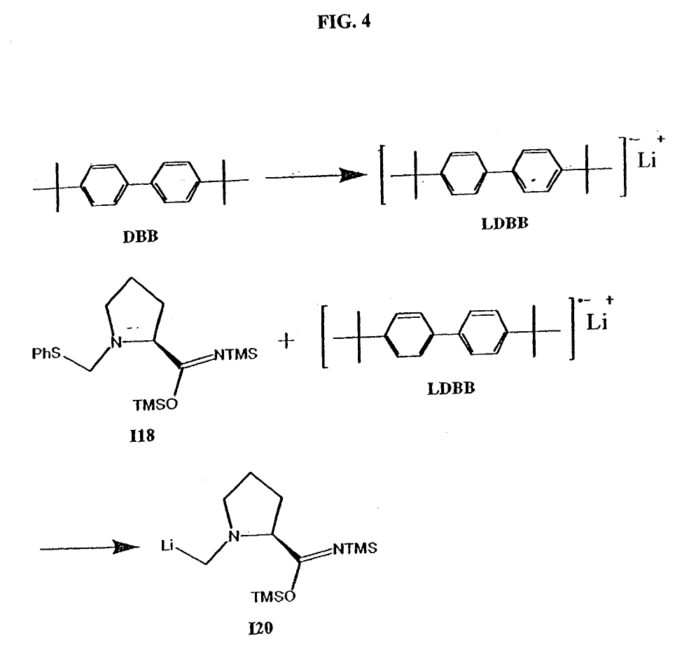

Certain specific intermediates useful for preparing compounds of formula I and II include compounds I12, I18, I19, I20, I21, I22, I25, I26, and I27, which are shown in FIGS. 2, 3, 4, 6, and 9.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion or as addition salts with amines, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compounds of formula I or II can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyiparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The ability of a compound of the invention to act as an inhibitor of HIV-1 protease may be determined using pharmacological models which are well known to the art, such as tnose described by A. D. Pivazyan et al., Biochem. Pharma, 2000, 60, 927. Compounds I24, I28a and I28b, described below, were found to be inhibitors of HIV-1 protease.

The invention will now be illustrated by the following non-limiting Examples.

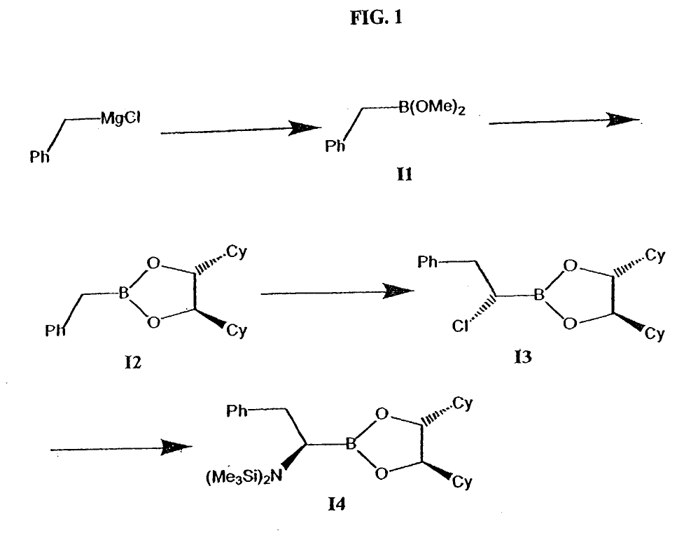

Synthesis of [2(1R),4R,5R]-4,5-dicyclohexyl-2-[1-bis(trimethylsilyl)amino-2-phenylethyl)-1,3,2-dioxaborolane (I4, FIG. 1) Cy=cyclohexyl Ph=phenyl.

Dimethyl benzylboronate (I1) was synthesized from benzyl magnesium chloride and trimethylborate at −78° C. and subsequent addition of methanesulfonic acid. Dimethyl benzylboronate (I1) was purified by fractional distillation. As an alternative method it was transformed into the corresponding pinacol ester, which was purified by column chromatography using a pentane/ether mixture as the solvent.

Dimethyl benzylboronate was transesterified to the corresponding R,R-DICHED (short for [(R,R)-1,2-dicylohexyl-1,2-ethanediol] ester, (4R,5R)-4,5-dicyclohexyl-2-(phenylethyl)-1,3,2-dioxaborolane (I2) in ether with optically pure [(R,R)-1,2-dicyclohexyl-1,2-ethanediol] (Matteson, D. S. et al., J. Am. Chem. Soc., 90, 7261-7266 (1968)). Compounds I1 and I2 are somewhat unstable in air but stable if kept under argon atmosphere.

(4R,5R)-4,5-Dicyclohexyl-2-(phenylethyl)-1,3,2-dioxaborolane (I2) was homologated at −100° C. with (dichloromethyl) lithium and anhydrous zinc dichloride resulting in the α-chloroboronic ester [2(1S),4R,5R]-4,5-dicyclohexyl-2-(1-chloro-2-phenylethyl)-1,3,2-dioxaborolane (I3) (Matteson, 1989). Compound I3 is unstable on silica and highly water and air sensitive. Methods for the directed chiral synthesis of certain α-chloroboronic esters are known. The α-chlorine can be easily displaced with alkyllithiums, Grignard reagents, lithium amides, alkoxides, and other groups in a very high stereospecific manner.

[2(1S),4R,5R]4,5-Dicyclohexyl-2-(1-chloro-2-phenylethyl)-1,3,2-dioxaborolane (I3) treated with lithium hexamethyldisilazane at −78° C. gave the silylated ester [2(1R),4R,5R]-4,5-dicyclohexyl-2-[1-bis(trimethylsilyl)amino-2-phenylethyl]-1,3,2-dioxaborolane (I4) (Matteson, D. S. et al., J. Am. Chem. Soc., 103, 5241-5242 (1981); Matteson, D. S. et al., Organometallics, 3, 1284-1288 (1984); and Matteson, D. S., Chemtech, 29, 6-14 (1999)). The major obstacle in the synthesis of boron-modified phenylalanine is the tendency of α-amino boronic esters to deboronate. This problem was solved by first synthesizing silylated amido boronic esters and then acylating them after desilylation. Compound I4 is highly water and air sensitive; therefore, extreme caution must be taken when handling it.

Synthesis of N-benzenethiomethyl L-proline (trimethylsilyloxy-methylene trimethylsilyl imine (I18, FIG. 2).

Compound I18 was obtained by the silylation of N-benzenethiomethyl L-proline amide (I17). N-Benzenethiomethyl L-proline amide (I17) was made at 0° C. from commercial L-proline amide in the presence of aqueous formaldehyde and benzenethiol. I17 was obtained in high yield and high purity. I17 was silylated using either chlorotrimethylsilane (method A) or hexamethyldisilazane (method B) as the silylating agent.

In method A N-benzenethiomethyl L-proline amide (I17) was reacted at 40° C. with chlorotrimethylsilane and triethylamine in the presence of sodium iodide in THF. After vacuum filtering sodium iodide and triethylamine hydrochloride and concentration the moisture sensitive solid, I18 was obtained. Di-silylation was achieved in approximately 76%, after several days.

In method B instead of chlorotrimethylsilane, hexamethyldisilazane was used. N-Benzenethiomethyl L-proline amide (I17) was reacted at 100° C. with hexamethyldisilazane. Approximately 59% di-silylation was obtained after 48 hours.

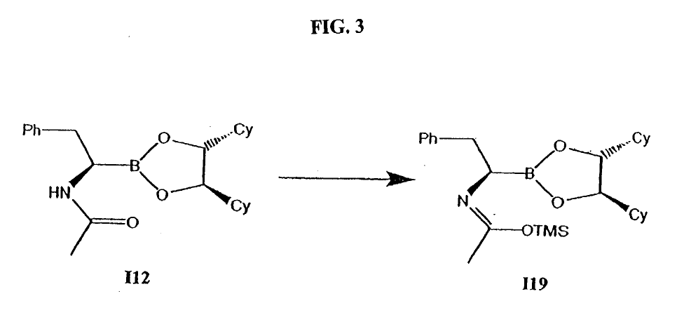

Synthesis of [2(1R),4R,5R]-4,5-dicyclohexyl-2-[1-(trimethylsilyloxyethylene)-imino-2-phenylethyl]-1,3,2-dioxaborolane (I19, FIG. 3).

For the synthesis of I19 the similar silylation procedure was followed as in method A for I18. [2(1R),4R,5R]-4,5-Dicyclohexyl-2-(1-acetamido-2-phenylethyl)-1,3,2-dioxaborolane (I12) was reacted at 40° C. with chlorotrimethylsilane and triethylamine in the presence of sodium iodide in THF. After vacuum filtering out sodium iodide and triethylamine hydrochloride the highly moisture sensitive [2(1R),4R,5R]4,5-dicylohexyl-2-[1-(trimethylsilyloxyethylene)-imino-2-phenylethyl]-1,3,2-dioxaborolane (I19) was obtained. Silylation took place approximately 72%, after 96 hours. Letting the reaction run longer did not improve the silylation yield. Using hexamethyldisilazane as silylating agent, as in method B for the synthesis of compound I18, no silylation was observed, the starting materials were recovered.

Lithiation of N-benzenethiomethyl L-proline (trimethylsilyloxy-methylene) trimethylsilyl imine (I18).

N-Benzenethiomethyl L-proline (trimethylsilyloxymethylene) trimethylsilyl imine (I18) was lithiated with lithium 4,4-di-tert-butylbiphenylide (LDBB). LDBB was prepared at 0° C. by the addition of lithium ribbon to a solution of di-tert-butylbiphenyl (DBB) in THF. After its formation the LDBB solution in THF was added at −100° C. to N-benzenethiomethyl L-proline (trimethylsilyloxymethylene) trimethylsilyl imine (I18).

After 5-10 minutes of stirring the dark green-blue color of the radical anion turned yellow suggesting the formation of N-lithiomethyl L-proline (trimethylsilyloxymethylene) trimethylsilyl imine (I20). After the reaction mixture turned yellow the reaction was quenched with D₂O and it was studied with NMR; only approximately 55% lithiation was observed. Previously, lithiation of R₂N—CH₂—SPh to R₂N—CH₂—Li has been used successfully, but usually with simple, unhindered tertiary amines. To improve the lithiation yield and further optimize reaction conditions a higher excess of LDBB solution can be used and/or the mixture can be stirred longer.

Synthesis of α-amido borinic acids.

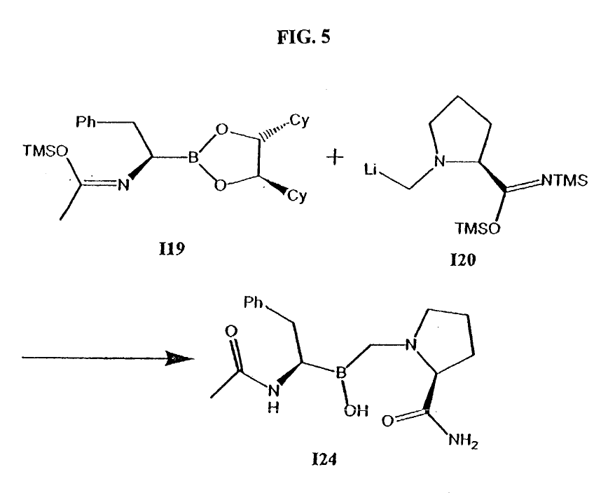

Synthesis of [(1R)-(1-acetamido-2-phenylethyl)]-B-methylene-L-proline amide borinic acid (I24, FIG. 5.

Borinic acids, compounds in which two carbons and one oxygen are bonded to boron, are a little studied class of compounds. The properties of borinic acids lie between those of boronic acids and trialkylboranes. The α-amido borinic acids synthesized herein contain a (1-acylamido-2-phenylethyl)boron group linked through a methylene spacer to the nitrogen atom of a proline unit. In the search for the best reaction conditions [(1R)-(1-acetamido-2-phenylethyl)]-B-methylene-L-proline amide borinic acid (I24) was synthesized using three different methods.

In method A I24 was prepared by first reacting [2(1R),4R,5R]4,5-dicyclohexyl-2-[1-(trimethylsilyloxyethylene)-imino-2-phenylethyl]-1,3,2-dioxaborolane (I19) at −100° C. with the previously synthesized N-lithiomethyl L-proline (trimethylsilyloxymethylene) trimethylsilyl imine (I20) in THF. After 1 hour of stirring chlorotrimethylsilane was added and the bath temperature was allowed to rise to room temperature. After 16 hours of further stirring methyl alcohol was added. After 1 hour of further stirring the reaction mixture was acidified with hydrochloric acid solution to pH=2, and the two phases were separated. The acidic aqueous phase was extracted with diethyl ether. Concentration of the acidic aqueous phase yielded the crude [(1R)-(1-acetamido-2-phenylethyl)]-B-methylene-L-proline amide borinic acid (I24).

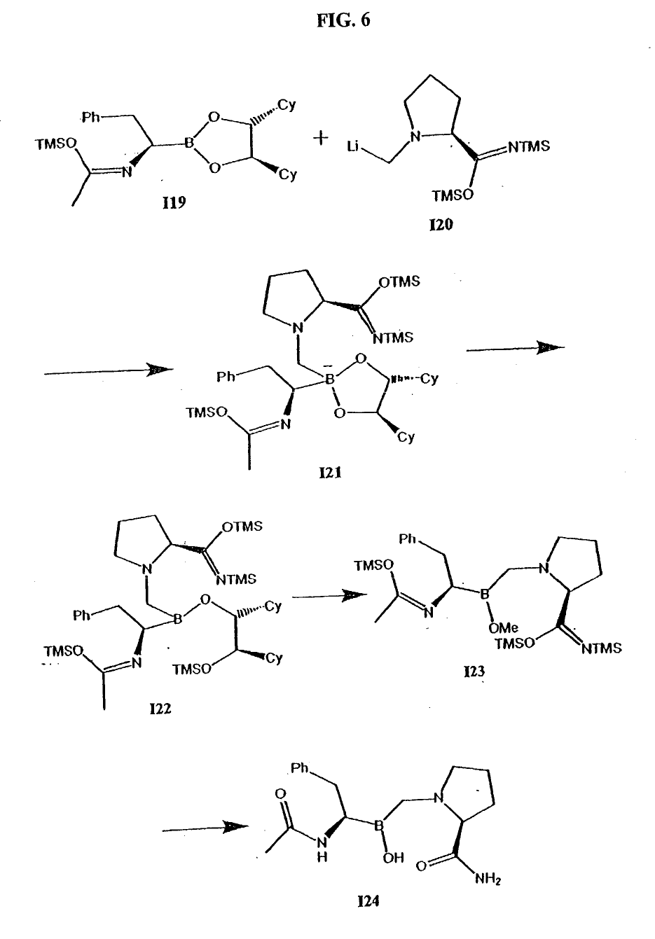

The capture of the lithiated species I20 by I19 is expected to take place fast at low temperature. The resulting, proposed intermediate after coupling I19 with I20 is the tetracoordinate boron species I21 (FIG. 6). By the addition of chlorotrimethylsilane, silylation and removal of one end of [(R,R)-1,2-dicyclohexyl-1,2-ethanediol] takes place, resulting in the tricoordinate boron species I22.

The silylated analogue of [(R,R)-1,2-dicyclohexyl-1,2-ethanediol] is completely removed by methyl alcohol, yielding borinic ester I23 (FIG. 6). Acidification yields the desired [(1R)-(1-acetamido-2-phenylethyl)]-B-methylene-L-proline amide borinic acid (I24). The suggested intermediates I21, I22, and I23 were not separated, purified, and characterized. A 160 MHz ¹¹B-NMR spectrum taken of an aliquot of I21 indicated a peak at 5.58 ppm, which corresponds to a tetracoordinate boron species. Furthermore, a 160 MHz ¹¹B-NMR spectrum taken of an aliquot of I23 indicated two peaks at 15.42 and 21.83 ppm, both of which correspond to tricoordinate boron species.

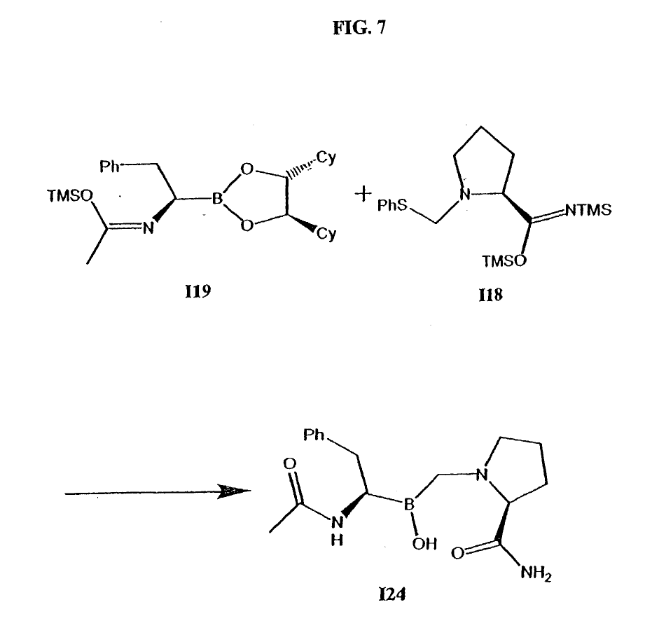

In method B (FIG. 7) I24 was synthesized by first making N-lithiomethyl L-proline (trimethylsilyloxymethylene) trimethylsilyl imine (I20) from N-benzenethiomethyl L-proline (trimethylsilyloxymethylene) trimethylsilyl imine (I18) in situ, in the presence of [2(1R), 4R, 5R]-4,5-dicyclohexyl-2-[1-(trimethylsilyloxyethylene)-imino-2-phenylethyl]-1,3,2-dioxa-borolane (I19) at −100° C. The dark green-blue color of the radical anion turned yellow approximately 10 min after its addition, suggesting that lithiation had taken place. Chlorotrimethylsilane and methyl alcohol were added and workup was done following the same procedure as in method A.

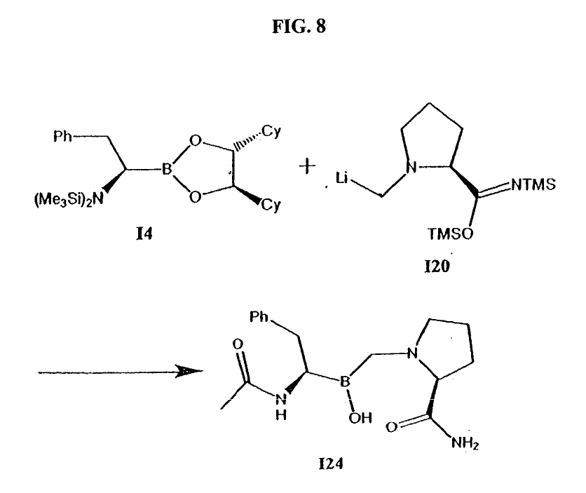

In method C (FIG. 8) I24 was prepared by first reacting [2(1R),4R,5R]-4,5-dicyclohexyl-2-[1-bis(trimethylsilyl)amino-2-phenylethyl]-1,3,2-dioxaborolane (I4) at −1 00° C. with the previously synthesized N-lithiomethyl L-proline (trimethylsilyloxymethylene) trimethyl-silylimine (I20) in THF. After 1 hour of stirring chlorotrimethylsilane was added and the bath temperature was allowed to rise to room temperature. After 16 hours of further stirring methyl alcohol was added. After 10 minutes of further stirring the reaction mixture was cooled down to −78° C. and acetic anhydride and acetic acid were added. The ice bath was removed after one hour and the bath temperature was allowed to rise to room temperature. After 24 hours of further stirring the reaction mixture was acidified with hydrochloric acid solution to pH 2, which was followed by the same workup as in methods A and B. Concentration of the acidic water phase yielded the crude [(1R)-( 1-acetamido-2-phenylethyl)] -B-methylene-L-proline amide borinic acid (I24).

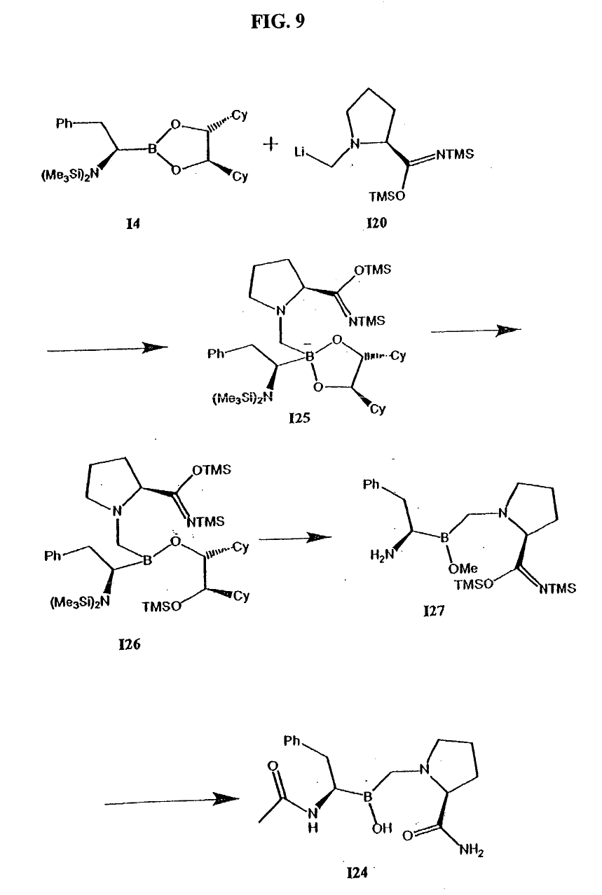

Coupling of I4 with I20 (FIG. 9) provides the tetracoordinate boron species I25. Chlorotrimethylsilane silylation and followed by removal of one end of [(R,R)-1,2-dicyclohexyl-1,2-ethanediol] provides the tricoordinate boron species I26. The silylated analogue of [(R,R)-1,2-dicyclohexyl-1,2-ethanediol] is completely removed by methyl alcohol, yielding a-amino borinic ester I27. Acylation with acetic anhydride and acetic acid and further acidification with hydrogen chloride yields the desired [(1R)-(1-acetamido-2-phenylethyl)]-B-methylene-L-proline amide borinic acid (I24). The intermediates I25, I26, and I27 were not separated, purified, or characterized.

The major advantage of method C is that it also allows the direct introduction of protected peptide chains previously prepared with the automated peptide synthesizer. Intermediate I27 can be acylated with different protected peptide chains in the presence of dicyclohexylcarbodiimide (DCC) or other coupling reagents; therefore, many peptide analogues can be fairly easily prepared. Furthermore, since acylation takes place later in the procedure bulkyness and increased functionality does not interfere with the process of coupling of I4 and I20.

Stability studies in buffer solutions have shown that I24 is stable in acidic and neutral media, but quickly decomposes under basic conditions. Furthermore, we attempted to separate the crude product on HPLC using a C₁₈ reverse phase column and water/acetonitrile as the solvent. After gradient separation we found that the product was unstable under the HPLC conditions used. Using trifluoroacetic acid as part of the solvent mixture will acidify the media and might prevent decomposition.

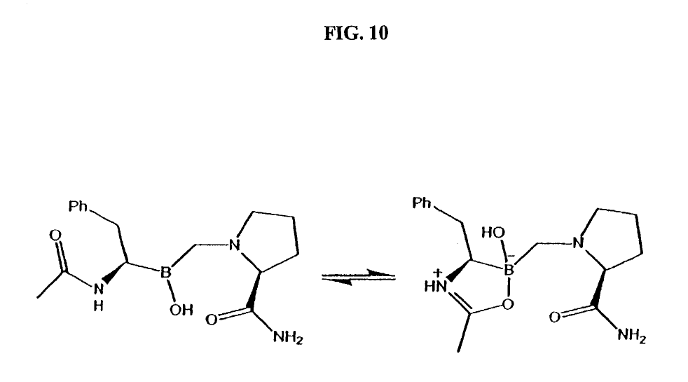

As studies suggest borinic acids are expected to be hydrolytically stable under physiological conditions and resistant to air oxidation. As expected, two dimensional HMQC, HMBC, and CIGAR experiments showed correlations between protons and carbons on both sides of the boron. The synthesized α-amido borinic acids can have two or three likely coordinated structures. The cyclic structures are expected to stabilize the α-amido borinic acids without interfering with binding to the enzyme. These likely structures are illustrated in FIG. 10. The Spiro structure, for which two diastereomers are possible, is likely present in highly acidic media.

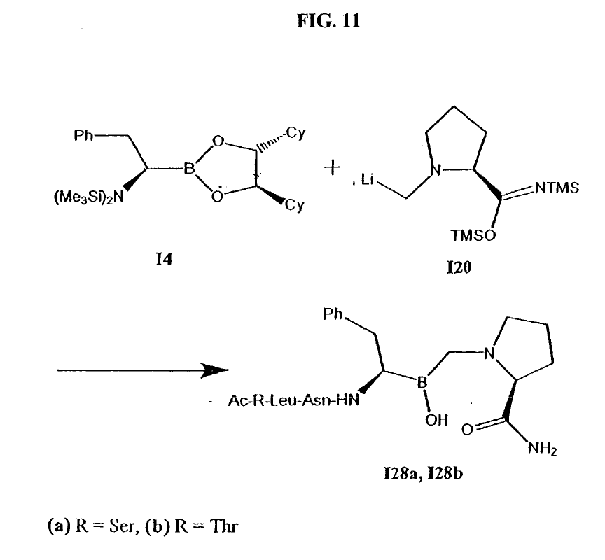

Synthesis of [(1R)-1(Ac-R-Leu-Asn)-2-phenylethyl]-B-methylene-L-proline amide borinic acids (I28a and I28b, FIG. 11).

For the synthesis of compounds I28a and I28b the same procedure was followed as for [(1R)-(1-acetamido-2-phenylethyl)]-B-methylene-L-proline amide borinic acid (I24), method C. The previously described α-amino borinic acid intermediate I27 was acylated at room temperature with Ac-Ser-Leu-Asn and Ac-Thr-Leu-Asn using dicyclohexylcarbodiimide (DCC), yielding the corresponding [(1R)-1(Ac-Thr-Leu-Asn)-2-phenylethyl]-B-methylene-L-proline amide borinic acid (I28a) and [(1R)-1-Ac-Thr-Leu-Asn)-2-phenylethyl]-B-methylene-L-proline amide borinic acid (I28b) respectively.

General Experimental. Tetrahydrofuran (THF) was distilled from benzophenone ketyl before use. Other anhydrous solvents, alkyllithiums, and Grignard reagents were supplied by Aldrich Chemical Company. All glassware was oven dried and cooled under argon before use. Liquid reagents and solutions were transferred using hypodermic syringes or double ended needles and were injected through rubber septa. All reactions were run under argon atmosphere.

### EXAMPLE 1

[(1R)-(1-Acetamido-2-phenylethyl)]-B-methylene-L-proline amide borinic acid (I24, FIGS. 6-9).

Method A: Lithium di-tert-butylbiphenylide was prepared by the addition of lithium ribbon (0.01 g, 2.10 mmol, 3.4 eq.) to a solution of di-tert-butylbiphenyl (DBB, 0.53 g, 1.98 mmol, 3.2 eq.) in THF (3.96 mL, calc. to 0.5 M sol.) at 0° C. The mixture was allowed to warm up to room temperature and it was stirred under inert atmosphere for 4-5 h. The dark green-blue color of the radical anion appeared within 10 min. After its formation, the lithium di-tert-butylbiphenylide solution was added to trimethylsilyl protected N-benzenethiomethyl L-proline amide I18 (0.23 g, 0.62 mmol, 1 eq.) at −100° C. The dark green-blue color of the radical anion turned to yellow 5-10 min. after the addition suggesting that lithiation had taken place. After 10 more minutes trimethyl silane protected [2(1R), 4R,5R]-4,5-dicyclohexyl-2-(1-acetamido-2-phenylethyl)-1,3,2-dioxaborolane I19 (0.29 g, 0.62 mmol, 1 eq.) was added at −100° C. Coupling of the two sides took place, resulting in the tetracoordinate boron compound intermediate I21. After 1 h. of stirring chlorotrimethylsilane (0.310 mL, 2.48 mmol, 4.0 eq.) was added. This resulted in the silylation; therefore, removal of one end of [(R,R)-1,2-dicyclohexyl-1,2-ethanediol] by braking one of the B—O bonds, yielding I22. The boron from being tetracoordinate became tricoordinate. The bath temperature was allowed to rise to room temperature and the reaction mixture was stirred under inert atmosphere for 16 hours. After 16 h. methyl alcohol (0.05 mL, 1.24 mmol, 2.0 eq.) was added. The addition of methyl alcohol resulted in the formation of the methyl ester analog I23 of the final borinic acid I24. The intermediates I21, I22, and I23 were not separated, purified, and characterized. After 1 h. of further stirring the reaction mixture was acidified with 1.0 M hydrochloric acid solution to pH 2, resulting in the final borinic acid I24. Before acidifying it the mixture was at pH 5-6. 1.0 M hydrochloric acid solution (15 mL) was diethyl ether (30 mL) were added. The water phase was washed two times with diethyl ether (2×30 mL). The aqueous phase was concentrated resulting in crude borinic acid I24 (0.06 g, 30 %, based on ¹H-NMR); 300 MHz ¹H-NMR (D₂O) δ 1.99 (m), 2.12 (m), 2.39 (m), 2.54 (m), 2.82 (m), 2.88 (s), 2.94 (s), 3.16 (m), 3.37 (m), 3.72 (m), 4.18 (t, J=9 Hz), 4.23 (t, J=9.6 Hz), 4.42 (m), 6.85 (s), 7.03 (s), 7.28 (m); 75 MHz ¹³C-NMR (D₂O) δ 16.8, 22.7, 23.1, 23.8, 28.5, 28.6, 29.8, 36.4, 41.1, 41.4, 56.9, 57.1, 59.8, 59.9, 68.3, 68.7, 126.7, 129.0, 129.1, 140.4, 170.7, 170.9, 171.6, 177.1; 160 MHz ¹¹B-NMR (D₂O) δ 9.77, 18.93; MALDI-MS Calcd. For C₁₆H₂₆BN₃O₃Li (M⁺+H₂Li): 326.22. Found: 326.21; C₁₆H₂₅BN₃O₃Li₂ (M⁺+HLi₂): 332.23. Found: 332.22; C₁₆H₂₅BN₃O₃LiNa (M⁺+HLiNa): 348.20. Found: 348.19.

Method B: Lithium di-tert-butylbiphenylide was prepared the same way as in method A, using the same amounts. After its formation, the lithium di-tert-butylbiphenylide solution was added to a mixture of trimethyl silane protected N-benzenethiomethyl L-proline amide I18 (0.23 g, 0.62 mmol, 1 eq.) and trimethyl silane protected [2(1R),4R,5R]-4,5-dicyclohexyl-2-(1-acetamido-2-phenylethyl)-1,3,2-dioxaborolane I19 (0.29 g, 0.62 mmol, 1 eq.) was added at −100° C. The dark green-blue color of the radical anion turned to yellow approximately 10 min. after the addition. After the in-situ lithiation, coupling, and 1 h. of further stirring chlorotrimethylsilane (0.62 mL, 4.95 mmol, 8.0 eq.) was added. Twice as much chlorotrimethylsilane was added as in method A in order to provide an even more acidic media. The rest of the procedure is the same as in method A (0.03 g, 15%, based on ¹H-NMR).

Method C: Lithium di-tert-butyibiphenyiide was prepared by the addition of lithium ribbon (0.02 g, 3.09 mmol, 5.0 eq.) to a solution of di-tert-butylbiphenyl (DBB, 0.79 g, 2.97 mmol, 4.8 eq.) in THF (5.94 mL, calc. to 0.5 M sol.) at 0° C. The mixture was let to warm up to room temperature and it was stirred under inert atmosphere for 4-5 h. The dark green-blue color of the radical anion appeared within 10 min. After its formation, the lithium di-tert-butylbiphenylide solution was added to trimethyi silane protected N-benzenethiomethyl L-proline amide I18 (0.23 g, 0.62 mmol, 1 eq.) at −100° C. The dark green-blue color of the radical anion turned to yellow 5-10 min. after the addition, suggesting that lithiation has taken place. After 10 more minutes [2(1R,),4R,5R]-4,5-dicyclohexyl-2-[1-bis(trimethylsilyl)amino-2-phenylmethyl)-1,3,2-dioxaborolane (I4) (0.31 g, 0.62 mmol, 1 eq.) was added at −100° C. Coupling of the two sides took place, resulting in the tetracoordinate boron compound intermediate I25. After 1 h. of stirring chlorotrimethylsilane (0.78 mL, 6.19 mmol, 10.0 eq.) was added. This resulted in the silylation; therefore, removal of one end of [(R,R)-1,2-dicyclohexyl-1,2-ethanediol] by braking one of the B—O bonds, yielding I26. The boron from being tetracoordinate became tricoordinate. The bath temperature was allowed to rise to room temperature and the reaction mixture was stirred under inert atmosphere for 16 h. After 16 h. methyl alcohol (0.08 mL, 1.98 mmol, 3.2 eq.) was added. The addition of methyl alcohol resulted in the formation of the methyl ester I27. After 10 min. of further stirring the reaction mixture was cooled down to −78° C., at which temperature acetic anhydride (0.17 nL, 1.86 mmol, 3 eq.) was added. The addition of acetic anhydride was immediately followed by the addition of acetic acid (0.04 mL, 0.74 mmol, 1.2 eq.). The ice-bath was removed after one hour and the reaction was further run under inert atmosphere, at room temperature for 24 h. The suggested intermediates I25, I26, and I27 were not separated, purified, and characterized. The same workup procedure was followed as in methods A and B (0.04 g, 20%, based on ¹H-NMR).

The intermediate compounds utilized in the above procedures were prepared as follows.

a. (4R,5R)-4,5-Dicyclohexyl-2-(phenylethyl)-1,3,2-dioxaborolane (I2). Dimethyl benzylboronate (I1) (15.00 g, 91.45 mmol) was mixed with [(R,R)-1,2-dicyclohexyl-1,2-ethanediol] (20.70 g, 91.45 mmol, 1 eq.) in diethyl ether (120 mL) at room temperature. The reaction mixture was stirred under inert atmosphere for 15 h. Removal of solvent at reduced pressure yielded oily I2 (29.84 g, 100%); 300 MHz ¹H-NMR (CDCl₃) δ 0.87-17.4 (m, 22H), 2.33 (s, 2H), 3.82 (d, J=4.5 Hz, 2H), 7.03-7.24 (m, 5H); 75 MHz ¹³C-NMR (CDCl₃) δ 19.2 (broad, C—B), 25.7, 25.8, 26.3, 27.2, 28.1, 42.6, 83.3, 124.6, 128.0, 128.8, 138.6, 160 MHz ¹¹B-NMR (CDCl₃) δ 32.2. HRMS Caled. for C₂₁H₃₁BO₂: 326.2417. Found: 326.2418. Anal. Calcd. for C₂₁H₃₁BO₂: C, 77.30; H, 9.58; B, 3.31. Found: C, 77.10; H, 9.60; B, 3.15.

b. [2(1S),4R,5R]-4,5-Dicyclohexyl-2-(1-chloro-2-phenylethyl)-1,3,2-dioxaborolane (I3). (Dichloromethyl) lithium was made by dropwise addition of butyllithium solution (35.91 mL, 57.46 mmol, 1.25 eq., 1.6 M sol. in hexanes) at −100° C. into a stirring solution of dichloromethane (8.89 mL, 137.91 mmol, 3 eq.) in THF (275.82 mL, calc. to 0.5 M sol.). After 5 minutes stirring (4R,5R)-4,5-dicyclohexyl-2-(phenylmethyl)-1,3,2-dioxaborolane (I2) (15.00 g, 45.97 mmol) solution in THF (10 mL) was added through cannula followed by zinc dichloride (68.95 mL, 68.95 mmol, 1.5 eq., 1.0 M solution in diethyl ether). The bath temperature was allowed to rise to room temperature and the reaction mixture was stirred under inert atmosphere for 24 h. Solvents were removed under reduced pressure and diethyl ether (100 mL) was added. The diethyl ether solution was washed three times with ammonium chloride solution and the ether phase was dried over anhydrous magnesium sulfate and filtered. Removal of solvent at reduced pressure yielded clear, viscous liquid I3 (16.88 g, 98%); 300 MHz ¹H-NMR (CDCl₃) δ 0.79-1.83 (M, 22H), 3.13 (m, 2H), 3.64 (t, J=8.4 Hz, 1H), 3.91 (d, J=5.1 Hz, 2H), 7.24 (m, 5H); 75 MHz ¹³C-NMR (CDCl₃) δ 25.7, 25.8, 26.3, 27.0, 27.9, 40.5, 42.6, 83.9, 126.6, 128.2, 129.0, 138.2. HRMS Calcd. for C₂₂H₃₂BClO₂: 374.2184. Found: 374.2187. Anal Calcd. for C₂₂H₃₂BClO₂: C, 70.51; H, 8.54; B, 2.88; Cl, 9.46. Found: C, 70.66; H, 8.48; B, 2.60; Cl, 9.32.

c. [2(1R),4R,5R]-4,5-Dicyclohexyl-2-[1-bis(trimethylsilyl)amino-2-phenylethyl]-1,3,2-dioxaborolane (I4). Lithiohexamethyldisilazane was made by dropwise addition of n-butyllithium (24.34 mL, 38.95 mmol, 1.01 eq., 1.6 M sol. in hexanes) at −78° C. to a stirring solution of hexamethyldisilazane (8.34 mL, 39.55 mmol, 1.02 eq.) in THF (77.11 mL, calc. to 0.5 M sol.). After 5 minutes of stirring a solution of [2(1S),4R,5R]-4,5-dicyclohexyl-2-(1-chloro-2-phenylmethyl)-1,3,2-dioxaborolane (I3) (14.45 g, 38.55 mmol) in THF (8 mL) was added through cannula. The bath temperature was allowed to rise to room temperature and the reaction mixture was stirred under inert atmosphere for 24 h. Solvents were removed under reduced pressure, pentane was added, and filtered under inert atmosphere through celite. Removal of solvent at reduced pressure yielded yellow viscous liquid I4 (18.29 g, 95%); 300 MHz ¹H-NMR (CDCl₃) δ 0.15 (s, 18H), 0.7-1.9 (m, 22H), 2.66 (m, 1H), 2.85 (m, 1H), 3.02 (m, 1H), 3.72 (d, J=4.5 Hz, 2H), 7.19 (m, 5H); 75 MHz ¹³C-NMR (CDCl₃) δ 2.9, 25.8, 26.1, 26.4, 27.5, 28.8, 42.5, 43.1, 44.1 (broad, C—B), 83.9, 125.6, 127.8, 129.6, 141.3. HRMS Calcd. for C₂₈H₄₉BNO₂Si₂ (M⁺−1): 498.3395. Found: 498.3409. Anal Calcd. for C₂₈H₄₉BNO₂Si₂: C, 67.30; H, 10.09; B, 2.16; N, 2.80; Si, 11.24. Found: C, 67.17; H, 10.30; B, 1.56; N, 2.80; Si, 11.24.

d. [2( 1R),4R,5R]-4,5-Dicyclohexyl-2-( 1-acetamido-2-phenylethyl)-1,3.2-dioxa-borolane (I12). A solution of [2(1R),4R,5R]-4,5-dicyclohexyl-2-[1-bis(trimethylsilyl)amino-2-phenylmethyl]-1,3,2-dioxaborolane (I4) (12.00 g, 24.01 mmol) in THF (24.01 mL, calc. to 1.0 M sol.) was stirred at −78° C. during the dropwise addition of acetic anhydride (6.79 mL, 72.04 mmol, 3 eq.) followed by acetic acid (1.51 mL, 26.41 mmol, 1.1 eq.). The ice-bath was removed after one hour and the reaction was run under inert atmosphere, at room temperature for 24 h. Removal of solvent at reduced pressure followed by recrystallization from ethyl alcohol yielded white, crystalline solid I12 (8.39 g, 88%); m.p. 184-186° C.; 300 MHz ¹H-NMR (CDCl₃) δ 0.96-1.35 (m, 12H), 1.70 (m, 8H), 1.93 (d, J=12.3 Hz, 2H), 2.02 (s, 3H), 2.69 (t, J=12.3 Hz, 1H), 2.99 (m, 2H), 3.69 (d, J=6.3 Hz, 2H), 6.18 (s, 1H), 7.26 (m, 5H); 75 MHz ¹³C-NMR (CDCl₃) δ 19.0, 26.4, 26.5, 26.9, 28.7, 29.7, 37.4, 43.4, 45.0 (broad, C—B), 83.1, 126.3, 128.8, 128.9, 140.9, 174.6; 160 MHz ¹¹B-NMR (CDCl₃) δ 19.2. Anal. Calcd. for C₂₄H₃₆BNO₃: C, 72.54; H, 9.13; B, 2.72; N, 3.52. Found: C, 72.42; H, 9.16; B, 2.69; N, 3.76.

e. N-Benzenethiomethyl L-proline amide (I17). To the chilled (0° C.) L-proline amide (1.00 g, 8.76 mmol) 37% aqueous formaidehyde solution (1.31 mL, 17.52 mmol, 2 eq.) was added dropwise, through syringe. The water bath was removed and benzenethiol (1.79 mL, 17.52 mmol, 2 eq.) was added dropwise. The reaction was run under inert atmosphere, at room temperature for 24 h. The white, crystalline product I17 started precipitating as soon as the benzenethiol was added. Vacuum filtration and washing with cold water resulted in pure, crystalline product I17 (1.43 g, 69%); m.p. 114-117° C.; 300 MHz ¹H-NMR (CDCl₃) δ 1.78 (m, 2H), 1.94 (m, 1H), 2.19 (m, 1H), 2.86 (m, 1H), 3.05 (m, 1H), 3.49 (dd, J=10.2 Hz, J=9.9 Hz, 1H), 4.41 (d, J=12.9 Hz, 1H), 4.55 (d, J=12.9 Hz, 1H), 6.10 (s, 1H), 6.75 (s, 1H), 7.26 (m, 3H), 7.4 (m, 2H); 75 MHz ¹³C-NMR (CDCl₃) δ 24.8, 31.6, 52.2, 61.9, 64.1, 127.0, 129.2, 131.7, 136.4, 177.3. Anal. Calcd. for C₁₂H₁₆N₂SO: C, 60.99; H, 6.83; N, 11.86; S, 13.54. Found: C, 61.02; H, 6.78; N. 11.79: S, 13.69.

f. N-Benzenethiomethyl L-proline (trimethylsilyloxymethylene)-trimethylsilyl imine (I18).

Method A: To a solution of N-Benzenethiomethyl L-proline amide (I17) (7.06 g. 29.87 mmol) and sodium iodide (6.72 g, 44.81 mmol, 1.5 eq.) in THF (59.74 mL, calc. to 0.5 M sol.) chlorotrimethylsilane (8.34 mL, 65.72 mmol, 2.2 eq.) was added, followed by triethylamine (8.32 mL, 59.74 mmol, 2.0 eq.). The reaction was run under inert atmosphere, at 40° C. for 96 h. The product mixture was vacuum filtered to remove sodium iodide and triethylamine hydrochloride. Concentration under vacuum yielded moisture-sensitive off-white solid, trimethylsilane protected N-benzenethiomethyl L-proline (trimethylsilyloxy-methylene)-trimethylsilyl imine (I18) (8.64 g, crude 76%, mono-silylation took place 100% and di-silylation 78%); m.p. 91-98° C.; 300 MHz ¹H-NMR (CDCl₃) δ 0.17 (s, 18H), 1.79 (m, 3H), 2.15 (m, 1H), 2.83 (m, 1H), 3.04 (m, 1H), 3.41 (dd, J=9.9 Hz, J=4.8 Hz, 1H), 4.36 (d, J=12.9 Hz, 1H), 4.52 (d, J=12.9 Hz, 1H), 7.24 (m, 3H), 7.40 (m, 2H).

Method B: To N-Benzenethiomethyl L-proline amide (I17) (1.00 g, 4.23 mmol) hexamethyldisilazane (5.34 mL, 25.39 mmol, 6.0 eq.) was added at 100° C. The reaction was run under inert atmosphere, at 100° C. for 48 h. The product mixture was concentrated under vacuum to remove the excess hexamethyldisilazane, yielding moisture-sensitive yellow, highly viscous liquid trimethylsilane protected N-benzenethiomethyl L-proline (trimethylsilyloxymethylene)-trimethylsilyl imine (I18) (1.33 g, crude 83%, mono-silylation took place 100% and di-silylation 59%).

g. [2(1R),4R,5R]-4,5-Dicyclohexyl-2-[1-(trimethylsilyloxvmethylene)-imino-2-phenyl-ethyl]-1 3,2-dioxaborolane (I19). To a solution of [2(1R),4R,5R]-4,5-dicyclohexyl-2-(1-acetamido-2-phenylmethyl)-1,3,2-dioxaborolane (I12) (1.21 g, 3.06 mmol) and sodium iodide (0.50 g, 3.36 mmol, 1.1 eq.) in THF (15.28 mL, calc. to 0.2 M sol.) chlorotrimethylsilane (0.58 mL, 4.58 mmol, 1.5 eq.) was added, followed by triethylamine (0.53 mL, 3.82 mmol, 1.25 eq.). The reaction was run under inert atmosphere, at 40° C. for 96 h. The product mixture was vacuum filtered to remove sodium iodide and triethylamine hydrochloride. Concentration under vacuum yielded moisture-sensitive white solid [2(1R), 4R,5R]-4,5-dicyclohexyl-2-[1-(trimethylsilyloxymethylene)-imino-2-phenylethyl]-1,3,2-dioxaborolane (I19) (1.26 g, crude 88%, silylation took place 72%); m.p. 151-157° C.; 300 MHz ¹H-NMR (CDCl₃) δ 0.17 (s, 9H), 0.85-1.89 (m, 22H), 2.02 (s, 3H), 2.69 (ddd, J=15.0 Hz, J=12 Hz, 3.0 Hz, 1H), 2.97 (m, 2H), 3.68 (d, J=5.7 Hz, 2H), 7.24 (m, 5H); 75 MHz ¹³C-NMR (CDCl₃) δ 1.6, 18.9, 26.4, 26.5, 26.9, 28.7, 29.7, 37.4, 43.4, 83.0, 126.2, 128.7, 128.8, 141.0, 174.7; 160 MHz ¹¹B-NMR (CDCl₃) δ 18.77.

### EXAMPLE 2

[( 1R)-1-(Ac-Ser-Leu-Asn)-2-phenylethyl]-B-methylene-L-proline amide borinic acid (I28a, FIG. 11).

Lithium di-tert-butylbiphenylide was prepared by the addition of lithium ribbon (0.003 g, 0.51 mmol, 5.0 eq.) to a solution of di-tert-butylbiphenyl (DBB, 0.13 g, 0.48 mmol, 4.8 eq.) in THF (0.97 mL, caic. to 0.5 M sol.) at 0° C. The mixture was let to warm up to room temperature and it was stirred under inert atmosphere for 4-5 h. The dark green-blue color of the radical anion appeared within 10 min. After its formation, the lithium di-tert-butylbiphenylide solution was added to trimethyl silane protected N-benzenethiomethyl L-proline amide I18 (0.04 g, 0.10 mmol, I eq.) at −100° C. The dark green-blue color of the radical anion turned to yellow 5-10 min. after the addition suggesting that lithiation has taken place. After 10 more minutes [2(1R),4R,5R]-4,5-dicyclohexyl-2-[1-bis(trimethylsilyl)amino-2-phenylmethyl)-1,3,2-dioxaborolane (I4) (0.05 g, 0.10 mmol, 1 eq. was added at −100° C. After 1 h. of stirring chlorotrimethylsilane (0.13 mL, 1.01 mmol, 10.0 eq.) was added. The bath temperature was allowed to rise to room temperature and the reaction mixture was stirred under inert atmosphere for 16 h. After 16 h. methyl alcohol (0.01 mL, 0.32 mmol, 3.2 eq.) was added. After 10 min. of further stirring Ac-Ser-Leu-Asn (0.04 g, 0.10 mmol, 1.0 eq.) and then 1,3-dicyclohexylcarbodiimide, DCC (0.05 mL, 0.30 mmol, 3 eq., 1.0 M solution in dichloromethane) were added. The reaction was further run under inert atmosphere, at room temperature for 24 h. After 24 h. the reaction mixture was acidified with 1.0 M hydrochloric acid solution to pH 2, resulting in the final tripeptide-borine acid I28a. Before acidifying it the mixture was at pH 5-6. 1.0 M hydrochloric acid solution 15 mL) and diethyl ether (30 mL) were added. The water phase was washed two times with diethyl ether (2×30 mL). The aqueous phase was concentrated resulting in crude I28a (0.03 g, 56% based on ¹H-NMR); 300 MHz ¹H-NMR (D₂O) δ 0.89 (d, J=16.5 Hz), 1.43 (m), 1.53 (s), 1.59 (m), 2.10 (m), 2.46 (m), 2.60 (m), 2.76 (s), 2.83 (m), 2.94 (s), 3.41 (m), 3.59 (m), 3.78 (m), 4.23 (t, J=9.3 Hz), 4.41 (m), 6.95 (s), 7.12 (s), 7.37 (m); 160 MHz ¹¹B-NMR (D₂O) δ 19.16; MALDI-MS Calcd. for C₂₉H₄₆N₇O₈Bli (M⁺+Li): 638.36. Found: 638.26.

### EXAMPLE 3

[(1R)-1-(Ac-Thr-Leu-Asn)-2-phenylethyl]-B-methylene-L-proline amide borinic acid (I28b, FIG. 11).

Lithium di-tert-butylbiphenylide was prepared by the addition of lithium ribbon (0.001 g, 0.21 mmol, 5.0 eq.) to a solution of di-tert-butylbiphenyl (DBB, 0.05 g, 0.20 mmol, 4.8 eq.) in THF (0.39 mL, calc. to 0.5 M sol.) at 0° C. The mixture was let to warm up to room temperature and it was stirred under inert atmosphere for 4-5 h. The dark green-blue color of the radical anion appeared within 10 min. After its formation, the lithium di-tert-butylbiphenylide solution was added to trimethyl silane protected N-benzenethiomethyl L-proline amide I18 (0.01 g, 0.04 mmol, 1 eq.) at −100° C. The dark green-blue color of the radical anion turned to yellow 5-10 min. after the addition suggesting that lithiation has taken place. After 10 more minutes [2(1R),4R,5R]-4,5-dicyclohexyl-2-[1-bis(trimethylsilyl)amino-2-[phenylethyl)-1,3,2-dioxaborolane (I4) (0.02 g, 0.04 mmol, 1 eq.) was added at −100° C. After 1 h. of stirring chlorotrimethylsilane (0.06 mL, 0.49 mmol, 12.0 eq.) was added. The bath temperature was allowed to rise to room temperature and the reaction mixture was stirred under inert atmosphere for 16 hours. After 16 h. methyl alcohol (0.005 mL, 0.13 mmol, 3.2 eq.) was added. After 10 min. of further stirring Ac-Thr-Leu-Asn (0.016 g, 0.04 mmol, 1.0 eq.) and then 1,3-dicyclohexylcarbodiimide, DCC (0.03 mL, 0.16 mmol, 4 eq., 1.0 M solution in dichloromethane) were added. The reaction was further run under inert atmosphere, at room temperature for 24 h. After 24 h. the reaction mixture was acidified with 1.0 M hydrochloric acid solution to pH 2, resulting in the final tripeptide-borinic acid I28b. Before acidifying it the mixture was at pH 5-6. 1.0 M hydrochloric acid solution (15 mL) and diethyl ether (30 mL) were added. The water phase was washed two times with diethyl ether (2×30 mL). The aqueous phase was concentrated, resulting in crude I28b (0.02 g, 88% based on ¹H-NMR); 300 MHz ¹H-NMR (D₂O) δ 0.85 (d, J=13.2 Hz), 1.53 (m), 2.02 (m), 2.41 (m), 2.53 (m), 2.89 (s), 3.36 (m), 3.71 (m), 4.17 (t, J=8.4 Hz), 4.36 (m), 7.37 (m); 160 MHz ¹¹B-NMR (D₂O) δ 19.18.

### EXAMPLE 4

The following illustrate representative pharmaceutical dosage forms, containing a compound of the invention (‘Compound X’), for therapeutic or prophylactic use in humans.

| (i) Tablet 1 | mg/tablet |
|---|---|
| ‘Compound X’ | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| ‘Compound X’ | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| ‘Compound X’ | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| ‘Compound X’ (free acid form) | 1.0 |
| Dibasic sodium phosphate | 12.0 |
| Monobasic sodium phosphate | 0.7 |
| Sodium chloride | 4.5 |
| 1.0 N Sodium hydroxide solution | q.s. |
| (pH adjustment to 7.0-7.5) | |
| Water for injection | q.s. ad 1 mL |

| (v) Injection 2 (10 mg/m1) | mg/ml |
|---|---|
| ‘Compound X’ (free acid form) | 10.0 |
| Monobasic sodium phosphate | 0.3 |
| Dibasic sodium phosphate | 1.1 |
| Polyethylene glycol 400 | 200.0 |
| 01 N Sodium hydroxide solution | q.s. |
| (pH adjustment to 7.0-7.5) | |
| Water for injection | q.s. ad 1 mL |

| (vi) Aerosol | mg/can |
|---|---|
| ‘Compound X’ | 20.0 |
| Oleic acid | 10.0 |
| Trichioromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of the formula I: 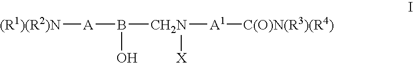
wherein:
A is the residue of an amino acid;
each A¹ is the residue of an amino acid; or N—A or N(X)A¹ independently represent a heterocyclic ring;
each R¹ is independently H, or a C₁-C₁₂ organic substituent; and R² is H, a C₁-C₁₂ organic substituent, or (R¹)(R³)N[—A¹—C(O)]ₙ; or R^{1 and R}² together with the nitrogen to which they are attached form a 5-7 membered heterocyclic ring, containing 1-3 N(R⁵), S or nonperoxide O;
X is H, or a C₁-C₁₂ organic substituent;
each R³ is independently H, or a C₁-C₁₂ organic substituent; and R⁴ is H, a C₁-C₁₂ organic substituent, or (R¹)(R³)N[—A¹—C(O)]ₙ; or R³ and R⁴ together with the nitrogen to which they are attached form a 5-7 membered heterocyclic ring, containing 1-3 N(R⁵), S or nonperoxide O;
n is 1-25; and
R⁵ is H, a C₁-C₁₂ organic substituent, or is absent;
or a pharmaceutically acceptable salt thereof.

2. The compound of claim 1 wherein A is the residue of an alpha-amino acid and A¹ is the residue of an alpha-amino acid.

3. The compound of claim 1 wherein A is the residue of an alpha-amino acid or A¹ is the residue of an alpha-amino acid.

4. The compound of claim 1 wherein the C₁-C₁₂ organic substituent is (C₁-C₆)alkyl, (C₆-C₁₀)aryl, (C₅-C₁₀)heteroaryl, (C₈-C₁₂)heteroaralkyl, (C₅-C₁₀)cycloalkyl, (C₂-C₄)acyl or ((C₂-C₄)alkyl)₃Si.

5. The compound of claim 1 wherein A is the residue of a naturally occurring alpha-amino acid.

6. The compound of claim 4 wherein A¹ is the residue of a naturally occurring alpha-amino acid.

7. The compound of claim 1 wherein R¹, X, R³ and R⁴ are H.

8. The compound of claim 1 wherein R¹, X, R³ or R⁴ is H.

9. The compound of claim 1 wherein X is (C₁-C₆)alkyl.

10. The compound of claim 1 wherein X is methyl.

11. The compound of claim 1 wherein n is 2-15

12. The compound of claim 1 wherein n is 3-10.

13. The compound of claim 1 wherien each of the organic substituents is independently selected from (C₁-C₆)alkyl, (C₂-C₆)alkenyl, (C₂-C₆)alkynyl, (C₆-C₁₀)aryl, (C₁-C₁₀)heteroaryl, (C₈-C₁₂)aralkyl, (C₂-C₁₂)heteroaralkyl, (C₅-C₁₀)cycloalkyl, (C₇-C₁₂)cycloalkyl(alkyl), (C₂-C₇)acyl and (C₁-C₄)alkyl)₃Si; wherein each organic substituent is optionally substituted with one or more (e.g. 1, 2, 3, or 4) substituents independently selected from OH, CN, NO₂, N(R^{a})(R^{b}), S(R^{a}), OR^{a}, —C(═O)OR^{a}, CO₂N(R^{a})(R^{b}), halo, and R^{a}C(═O)O—; and wherein each R^{a} and R^{b} is independently hydrogen or (C₁-C₆)alkyl.

14. The compound of claim 1 wherein A¹ is the residue of an amino acid.

15. The compound of claim 1 wherein R¹ is H and R² is (C₂-C₇)acyl.

16. The compound of claim 15 wherein R² is acetyl.

17. The compound of claim 1 wherein:
A is the residue of a naturally occurring or synthetic alpha-amino acid;
A¹ is the residue of a naturally occurring or synthetic amino acid; or N—A or N(X)A¹ independently represent a heterocyclic ring;
each R¹ is independently H, (C₁-C₆)alkyl, (C₆-C₁₀)aryl, (C₅-C₁₀)heteroaryl, (C₈-C₁₂)aralkyl, (C₈-C₁₂)heteroaralkyl, (C₅-C₁₀)cycloalkyl, (C₇-C₁₂)cycloalkylalkyl, (C₂-C₇)acyl or (C₁-C₄)alkyl)₃Si; and R² is H, (C₁-C₆)alkyl, (C₆-C₁₀)aryl, (C₅-C₁₀)heteroaryl, (C₈-C₁₂)aralkyl, (C₈-C₁₂)heteroaralkyl, (C₅-C₁₀)cycloalkyl, (C₇-C₁₂)cycloalkylalkyl, (C₂-C₇)acyl (C₁-C₄)alkyl)₃Si or (R¹)(R³)N[—A—C(O)]ₙ—; or R¹ and R² together with the nitrogen to which they are attached form a 5-7 membered heterocyclic ring, containing 1-3 N(R⁵), S or nonperoxide O;
each R³ is independently H, (C₁-C₆)alkyl, (C₆-C₁₀)aryl, (C₅-C₁₀)heteroaryl, (C₈-C₁₂)aralkyl, (C₈-C₁₂)heteroaralkyl, (C₅-C₁₀)cycioalkyl, (C₇-C₁₂)cycloalkylalkyl, (C₂-C₇)acyl or (C₁-C₄)alkyl)₃Si; and R⁴ is H, (C₁-C₆)alkyl, (C₆-C₁₀)aryl, (C₅-C₁₀)heteroaryl, (C₈-C₁₂)aralkyl, (C₈-C₁₂)heteroaralkyl, (C₅-C₁₀)cycloalkyl, (C₇-C₁₂)cycloalkylalkyl, (C₂-C₇)acyl (C₁-C₄)alkyl)₃Si or (R¹)(R³)N[—A—C(O)]ₙ—; or R³ and R⁴ together with the nitrogen to which they are attached form a 5-7 membered heterocyclic ring, containing 1-3 N(R⁵), S or nonperoxide O;
X is H (C₁-C₆)alkyl, (C₆-C₁₀)aryl, (C₅-C₁₀)heteroaryl, (C₈-C₁₂)aralkyl, (C₈-C₁₂)heteroaralkyl, (C₅-C₁₀)cycloalkyl, (C₇-C₁₂)cycloalkylalkyl, (C₂-C₇)acyl or (C₁-C₄)alkyl)₃Si;
n is 1-25;
R⁵ is absent or is H, (C₁-C₆)alkyl, (C₆-C₁₀)aryl, (C₅-C₁₀)heteroaryl, (C₈-C₁₂)aralkyl, (C₈-C₁₂)heteroaralkyl, (C₅-C₁₀)cycloalkyl, (C₇-C₁₂)cycloalkylalkyl, (C₂-C₇)acyl or (C₁-C₄)alkyl)₃Si;
or a pharmaceutically acceptable salt thereof.

18. The compound of claim 17 wherein R¹, X, R³ and R⁴ are H.

19. The compound of claim 17 wherein R¹, X, R³ or R⁴ is H.

20. The compound of claim 17 wherein R¹ is H and R² is (C₂-C₇)acyl.

21. The compound of claim 20 wherein R² is acetyl.

22. The compound of claim 16 or 21 wherein N(X)A¹ is pyrrolidin-2-yl or 4-hydroxyl-2-pyrrolidinyl.

23. The compound of claim 1 or 17 wherein A is CH(phenyl).

24. The compound of claim 1 or 17 wherein (R¹)(R³)N[—A—C(O)]ₙ is CH₃C(O)-Ser-Leu-Asn- or Ac-Thr-Leu-Asn and R¹ is H.

25. The compound of claim 1 wherein R³ and R⁴ are H.

26. The compound of claim 22 wherein N(X)A¹—C(O)N(R³)R⁴) is

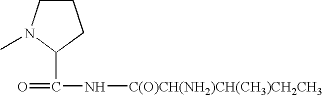

27. The compound of claim 1, 17, or 26 wherein R¹ is acetyl; R² is hydrogen; and A is a polypeptide residue comprising 2-10 amino acid residues.

28. The compound CH₃C(O)-Thr-Leu-Asn-Phe-B(OH)CH₂Pro-Ile; or a pharmaceutically acceptable salt thereof.

29. The compound CH₃C(O)-Leu-Asn-Phe-B(OH)CH₂-Pro-Ile; or a pharmaceutically acceptable salt thereof.

30. The compound CH₃C(O)-Asn-Phe-B(OH)CH₂-Pro-Ile; or a pharmaceutically acceptable salt thereof.

31. The compound CH₃C(O)-Ser-Leu-Asn-Phe B(OH)CH₂ProNH₂; or a pharmaceutically acceptable salt thereof.

32. The compound CH₃C(O)-Thr-Leu-Asn-Phe B(OH)CH₂ProNH₂; or a pharmaceutically acceptable salt thereof.

33. The compound CH₃C(O)-Phe B(OH)CH₂ProNH₂; or a pharmaceutically acceptable salt thereof.

34. A method of inhibiting the activity of a mammalian protease comprising contacting said protease with an effective inhibitory amount of a compound according to claim 1.

35. The method of claim 34 comprising administering the compound to a mammal afflicted with a condition that is ameliorated by protease inhibition.

36. The method of claim 35 wherein the protease is HIV-1 protease, and the condition is HIV infection.

37. A pharmaceutical composition comprising a compound of claim 1 and a pharmaceutical carrier.
